(19) United States
(12) Patent Application Publication
(10) Pub. No.: US 2026/0022084 A1
(43) Pub. Date: Jan. 22, 2026

(54) METHOD FOR SEPARATING AND PURIFYING MONOCYCLIC AROMATIC HYDROCARBONS FROM DIRECT COAL LIQUEFACTION OIL

(71) Applicant: TAIYUAN UNIVERSITY OF TECHNOLOGY, Taiyuan (CN)

(72) Inventors: Yuncai SONG, Taiyuan (CN); Xiaolong SHI, Taiyuan (CN); Jie FENG, Taiyuan (CN); Wenying LI, Taiyuan (CN)

(73) Assignee: TAIYUAN UNIVERSITY OF TECHNOLOGY, Taiyuan (CN)

(21) Appl. No.: 19/344,629

(22) Filed: Sep. 30, 2025

Related U.S. Application Data
(63) Continuation of application No. PCT/CN2024/144273, filed on Dec. 31, 2024.

(30) Foreign Application Priority Data
Jul. 22, 2024 (CN) 202410984058.8

(51) Int. Cl. C07C 7/00

(52) U.S. Cl. CPC C07C 7/005

(57) ABSTRACT

A method for separating and purifying monocyclic aromatic hydrocarbons from direct coal liquefaction oil is provided. The method is as follows: (1) performing fine fractionation and component analysis on the direct coal liquefaction oil, to determine a highest fraction of monocyclic aromatic hydrocarbons; (2) Removing light alkanes and cycloalkanes from the selected fraction by column chromatography; (3) separating toluene, monocyclic aromatic hydrocarbon mixtures and high-carbon alkanes through atmospheric and vacuum distillation column; (4) purifying ethylbenzene from a mixture of monocyclic aromatic hydrocarbons through extractive distillation column and (5) purifying p-xylene through solvent recovery column The separation method may fully utilize the compositional characteristics of direct coal liquefaction oil, to obtain two to three high-purity monomers of monocyclic aromatic hydrocarbons in a single processing step, without introducing additional chemical reaction steps, thereby demonstrating significant economic and environmental benefits.

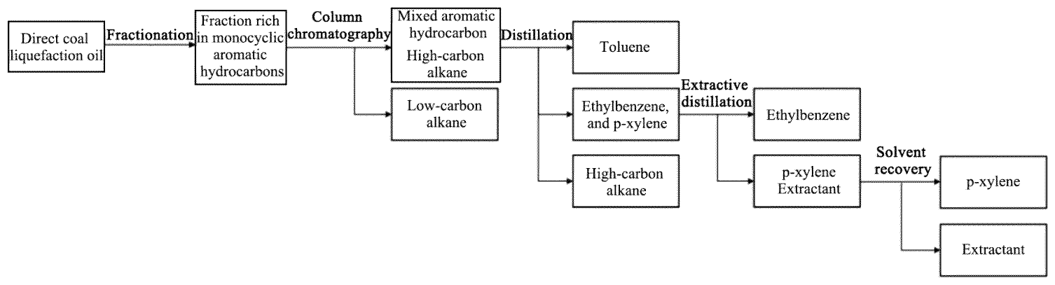

### CROSS REFERENCE TO THE RELATED APPLICATIONS

This application is a continuation application of International Application No. PCT/CN2024/144273, filed on Dec. 31, 2024, which is based upon and claims priority to Chinese Patent Application No. 202410984058.8, filed on Jul. 22, 2024, the entire contents of which are incorporated herein by reference.

### TECHNICAL FIELD

The present disclosure relates to the technical field of separation from direct coal liquefaction oil, and in particular to a method for separating and purifying monocyclic aromatic hydrocarbons from direct coal liquefaction oil.

### BACKGROUND

toluene, ethylbenzene and xylene are all basic chemical raw materials. In recent years, the demand for toluene, ethylbenzene and p-xylene in China has been increasing year by year. The raw material for producing p-xylene is usually petroleum-based naphtha, for example, direct distillation and secondary processing naphtha. However, with the obvious trend of densification and deterioration of exploiting crude oil, the supply of petroleum-based naphtha for producing toluene, ethylbenzene and p-xylene is tight, which is not enough to match the growing demand for toluene, ethylbenzene and p-xylene. Therefore, there is an urgent need for raw materials suitable for producing toluene, ethylbenzene and p-xylene.

In coal-based crude oil, the separation of hydrocarbons and the separation of xylene isomers from coal-based crude oil are of great significance. Direct coal liquefaction technology is one of the methods of coal to oil, and is the process of converting coal into liquid fuel. In the coal liquefaction oil obtained from this process, naphtha fraction accounts for about 15-30 wt %, and the fraction has a high aromatic content. Coal-based mixed aromatics with aromatics content of more than 80 wt % can be further obtained, which is very suitable for producing toluene, ethylbenzene and p-xylene.

At present, the process of separating and purifying p-xylene from coal-based crude oil is mainly based on catalytic conversion and separation. Catalytic conversion is to convert p-xylene into other compounds by catalytic reaction, thereby separating from other isomers. CN 103436288 A discloses a method for preparing aromatic hydrocarbons from coal tar naphtha, performing pretreatment and vacuum distillation on coal tar, coking, cracking or hydrotreating to obtain naphtha fraction, then performing pre-fractionation, pre-hydrotreating and reforming on the naphtha fraction to obtain a light aromatic hydrocarbon mixture, finally, performing separation and further isomerization on the light aromatic hydrocarbon mixture to obtain benzene and p-xylene. CN 106187671A discloses a method for separating p-xylene from coal direct liquefied naphtha, performing hydrotreating, reforming and aromatics extraction on direct coal liquefaction naphtha to obtain coal-based mixed aromatics, and then performing fractionation, alkylation, disproportionation and transalkylation, adsorption and separation on the coal-based mixed aromatics to obtain P-xylene with a purity of 99.5 wt %.

At present, the catalytic conversion method for the production of p-xylene from coal-based crude oil needs to be separated by reaction conversion, the control variables are complicated, additional chemical reaction steps may be introduced, and economic benefits and environmental impacts need to be considered comprehensively. Therefore, it is necessary to find a simpler process to realize the separation of p-xylene from coal-based oil, to make full use of direct coal liquefaction oil as raw material for the production of p-xylene, doing deep processing and high-value utilization of direct coal liquefaction products, thereby meeting the growing market demand for p-xylene products.

### SUMMARY

An objective of the present disclosure is to provide a method for separating and purifying monocyclic aromatic hydrocarbons from direct coal liquefaction oil, which is used to address the technical problem that the separation process of p-xylene in existing coal-based oil products is complicated and the economic and environmental benefits need to be considered comprehensively.

In order to achieve the above effects, the present disclosure adopts the following technical solutions.

The present disclosure provides a method for separating and purifying monocyclic aromatic hydrocarbons from direct coal liquefaction oil, including the following steps:
1) performing fine fractionation on the direct coal liquefaction oil, and analyzing components of obtained fraction sections;
2) selecting a fraction with a highest content of monocyclic aromatic hydrocarbons, and sequentially performing column chromatography, atmospheric and vacuum distillation and extractive distillation on fractions in the fraction section, to obtain a distillation product;
3) performing solvent recovery on the distillation product, to obtain a monocyclic aromatic hydrocarbon; and
the monocyclic aromatic hydrocarbon includes one or more of toluene, ethylbenzene, and p-xylene.

In some embodiments, in step 1), a reflux ratio of the fine fractionation is ≥3, a temperature range is ≤180° C., and a quantity of fractions is 8-15.

In some embodiments, in step 2), a stationary phase of the column chromatography is silica gel for chromatography and/or neutral aluminum oxide; and

when the stationary phase is a mixture of silica gel for chromatography and neutral aluminum oxide, a mass ratio of silica gel for chromatography to neutral aluminum oxide is (3-4):(2-3).

In some embodiments, in step 2), the mobile phase contains eluent 1 and eluent 2, and a volume ratio of eluent 1 and eluent 2 is 10:(3-5),
wherein, eluent 1 and eluent 2 are independently one or more of n-pentane, isopentane, petroleum ether, n-hexane, n-heptane, dichloromethane, ethyl acetate and chloroform.

In some embodiments, in step 2), after the column chromatography, the obtained column chromatography product is a mixture from which low-carbon alkanes and cycloalkanes have been removed.

In some embodiments, in step 2), the atmospheric and vacuum distillation is performed at least once, and the atmospheric and vacuum distillation product is a mixture from which high carbon alkanes have been removed.

In some embodiments, during the atmospheric and vacuum distillation, a column overhead temperature is independently 100-140° C., a column bottom temperature is independently 150-220° C., a quantity of trays is independently 20-40, and a reflux ratio is independently 2-5, a column overhead pressure is 0.10-0.15 MPa, and a column bottom pressure is 0.15-0.17 MPa.

In some embodiments, in step 2), the extractant for extractive distillation is one or more of polychlorobenzenes, anhydrides, esters, ketones, alcohols and pyridines.

In some embodiments, in step 3), during the extractive distillation, a column overhead temperature is 130-200° C., and a column bottom temperature is 130-200° C., a quantity of trays is 130-200, a reflux ratio is 35-45, a column overhead pressure is 0.10-0.15 MPa, and a column bottom pressure is 0.2-0.25 MPa.

In some embodiments, in step 3), during the solvent recovery, a column overhead temperature is 130-140° C., a column bottom temperature is 195-215° C., a quantity of trays is 24-30, a reflux ratio is 2-3, a column overhead pressure is 0.1-0.15 MPa, and a column bottom pressure is 0.17-0.2 MPa.

The beneficial effects of the present disclosure are as below:

By using the method of producing monocyclic aromatic hydrocarbons provided by the present disclosure, high-purity toluene, ethylbenzene and p-xylene can be produced by making full use of direct coal liquefaction oil resources. In particular, high-purity p-xylene can be produced by utilizing the composition characteristics of coal conversion resources, the method of producing p-xylene provided by the present disclosure avoids the additional chemical reaction steps introduced by catalytic conversion and the environmental problems caused by it, and the process is simple, economical and efficient. The present disclosure makes full use of direct coal liquefaction oil resources to alleviate the problem of insufficient supply of petroleum-based naphtha in China, and provides a new idea for making full use of energy.

### BRIEF DESCRIPTION OF THE DRAWINGS

FIGURE is a schematic diagram of the process flow for separating and purifying monocyclic aromatic hydrocarbons from direct coal liquefaction oil of the present disclosure.

### DETAILED DESCRIPTION OF THE EMBODIMENTS

The present disclosure provides a method for separating and purifying monocyclic aromatic hydrocarbons from direct coal liquefaction oil, including the following steps:
1) fine fractionation is performed on the direct coal liquefaction oil, and analyzing components of obtained fraction sections are analyzed;
2) a fraction with a highest content of monocyclic aromatic hydrocarbons is selected, and column chromatography, atmospheric and vacuum distillation and extractive distillation are sequentially performed on fractions in the fraction section, to obtain a distillation product;
3) solvent recovery is performed on the distillation product, to obtain a monocyclic aromatic hydrocarbon; and
the monocyclic aromatic hydrocarbon includes one or more of toluene, ethylbenzene, and p-xylene.

In the present disclosure, in step 1), a reflux ratio of the fine fractionation is ≥3, preferably ≥5, and further preferably ≥7; a temperature range is preferably ≤180° C., and a quantity of fractions is 8-15, preferably 9-12, and further preferably 10 fractions at temperatures including <80° C., 80-100° C., 100-110° C., 110-120° C., 120-130° C., 130-140° C., 140-150° C., 150-160° C., 160-170° C. and 170-180° C.

In the present disclosure, in step 2), a stationary phase of the column chromatography is silica gel for chromatography and/or neutral aluminum oxide, and preferably silica gel for chromatography and neutral aluminum oxide.

When the stationary phase is silica gel for chromatography and neutral aluminum oxide, a mass ratio of silica gel to neutral alumina is (3-4):(2-3), and preferably (3.2-3.8):(2.2-2.8), and further preferably (3.4-3.6):(2.4-2.6), and more preferably 3.5:2.5.

In the present disclosure, in step 2), the mobile phase contains eluent 1 and eluent 2, and a volume ratio of eluent 1 and eluent 2 is 10:(3-5), preferably 10:(3.5-4.5), and more preferably 10:4.

Wherein, eluent 1 and eluent 2 are independently one or more of n-pentane, isopentane, petroleum ether, n-hexane, n-heptane, dichloromethane, ethyl acetate and chloroform, preferably one or more of n-hexane, n-heptane, dichloromethane, ethyl acetate and chloroform, and further preferably n-hexane and dichloromethane.

In the present disclosure, in step 2), after the column chromatography, the obtained column chromatography product is a mixture from which light alkanes and cycloalkanes have been removed, preferably components rich in monocyclic aromatic hydrocarbons and from which low-carbon alkanes and cycloalkanes are removed, and more preferably components rich in C8 aromatics and from which light alkanes and cycloalkanes have been removed.

In the present disclosure, in step 2), the atmospheric and vacuum distillation is performed at least once, preferably not less than 2 times, and further preferably not less than 3 times, the atmospheric and vacuum distillation product is a mixture from which high carbon alkanes have been removed, preferably a mixture from which carbon seven aromatic hydrocarbons and high carbon alkanes have been removed, and more preferably a mixture of ethylbenzene and p-xylene.

In the present disclosure, during the atmospheric and vacuum distillation, a column overhead temperature is independently 100-140° C., preferably 110-130° C., and more preferably 120° C.; a column bottom temperature is independently 150-220° C., preferably 170-200° C., and more preferably 190° C.; a quantity of trays is independently 20-40 independently, preferably 25-35, and more preferably 30; and a reflux ratio is independently 2-5 independently, preferably 2.5-4.5, and more preferably 3; a column overhead pressure is 0.10-0.15 MPa, preferably 0.12-0.14 MPa, and more preferably 0.13 MPa; and a column bottom pressure is 0.15-0.17 MPa, and more preferably 0.16 MPa.

In the present disclosure, in step 2), the extractant for extractive distillation is one or more of polychlorobenzenes, anhydrides, esters, ketones, alcohols and pyridines, preferably one or more of polychlorobenzenes, anhydrides, and esters, and preferably polychlorobenzenes or anhydrides, and more preferably 1,2,4-trichlorobenzene.

In the present disclosure, in step 3), during the extractive distillation, a column overhead temperature is 130-200° C., preferably 150-180° C., and more preferably 160° C.; a column bottom temperature is 130-200° C., preferably 150-180° C., and more preferably 160° C.; a quantity of trays is 130-220, preferably 150-200, further preferably 170-190, and more preferably 180; a reflux ratio is 35-45, preferably 38-42, and further preferably 40; a column overhead pressure is 0.10-0.15 MPa, preferably 0.12-0.14 MPa, and more preferably 0.13 MPa; and a column bottom pressure is 0.2-0.25 MPa, preferably 0.22-0.24 MPa, and more preferably 0.23 MPa.

In the present disclosure, in step 3), during the solvent recovery, a column overhead temperature is 130-140° C., preferably 132-138° C., and more preferably 135° C.; a column bottom temperature is 195-215° C., preferably 200-210° C., and more preferably 205° C.; a quantity of trays is 24-30, preferably 25-28, and more preferably 27; a reflux ratio is 2-3, preferably 2.3-2.8, and more preferably 2.5; a column overhead pressure is 0.1-0.15 MPa, preferably 0.12-0.14 MPa, and further preferably 0.13 MPa; and a column bottom pressure is 0.17-0.2 MPa, and preferably 0.18 MPa.

In the following, the technical solutions provided by the present disclosure are described in detail in combination with the embodiments, but they cannot be understood as limiting the scope of protection of the present disclosure.

### Embodiment 1

A reflux ratio of the distilling tower was set to 5, the direct liquefaction oil of Xinjiang coal was cut out 10 fractions at temperatures including <80° C., 80-100° C., 100-110° C., 110-120° C., 120-130° C., 130-140° C., 140-150° C., 150-160° C., 160-170° C. and 170-180° C., after analyzing the composition of each fraction, the fraction with the highest content of monocyclic aromatic hydrocarbons was selected as 120-130° C.;
column chromatography was performed on the fractions in the 120-130° C. fraction (the lower layer of the stationary phase was 3.28 g silica gel for chromatography, the upper layer was 2.82 g neutral aluminum oxide, and the column size was 15*300 mm). Firstly, alkanes and cycloalkanes were completely removed by elution with 30 mL of n-hexane (eluent 1); followed by a fraction containing 5.12 wt % monocyclic aromatic hydrocarbons was obtained by elution with 9 mL of a mixed solvent (eluent 2) including dichloromethane and n-hexane (a volume ratio of 2:1) (in monocyclic aromatic hydrocarbons, toluene accounted for 24 wt %, ethylbenzene accounted for 26 wt %, and p-xylene accounted for 50 wt %).

A first atmospheric and vacuum distillation was performed on the column chromatography products to obtain toluene and a distillate at the bottom of the first tower; a second atmospheric and vacuum distillation was performed on the distillate at the first tower, to obtain mixed aromatics and a distillate at the second tower bottom; extractive distillation was performed on the distillate at the second tower bottom by using 1,2,4-trichlorobenzene, after ethylbenzene was obtained, and then the solvent recovery was performed through the solvent recovery tower, to obtain p-xylene.

Table 1 shows process conditions in Embodiment 1, and Table 2 shows product compositions of the tower top in Embodiment 1.

TABLE 1
Process conditions in Embodiment 1

| | First | Second | | |
|---|---|---|---|---|
| | atmospheric | atmospheric | | |
| | and vacuum | and vacuum | Extractive | Solvent |
| Process conditions | distillation | distillation | distillation | recovery |
| Quantity of trays | 33 | 21 | 207 | 30 |
| Reflux ratio | 2 | 15 | 44 | 2 |
| Column overhead | 110.4 | 138.9 | 135.5 | 137.9 |
| temperature/° C. | | | | |
| Column bottom | 173.9 | 177.5 | 163.1 | 197.7 |
| temperature/° C. | | | | |
| Column overhead | 0.11 | 0.11 | 0.11 | 0.11 |
| pressure/MPa | | | | |
| Column bottom | 0.17 | 0.16 | 0.23 | 0.17 |
| pressure/MPa | | | | |

TABLE 2
Composition of the overhead product from Embodiment 1

| | First | Second | | |
|---|---|---|---|---|
| | atmospheric | atmospheric | | |
| | and vacuum | and vacuum | Extractive | Solvent |
| | distillation | distillation | distillation | recovery |
| Composition | column | column | column | column |
| Toluene/wt % | 99.05 | 0.14 | 0.47 | 0.00 |
| Ethylbenzene/wt % | 0.53 | 30.28 | 97.62 | 0.52 |
| P-xylene/wt % | 0.42 | 58.59 | 1.91 | 99.29 |
| High-carbon | 0.00 | 10.98 | 0.00 | 0.19 |
| alkane/wt % | | | | |

### Embodiment 2

Different from Embodiment 1, in the present embodiment, the stationary phase of column chromatography was composed of 3.71 g silica gel for chromatography and 2.64 g neutral aluminum oxide; a dosage of eluent 2 was 12 mL, a content of monocyclic aromatic hydrocarbons in the fraction obtained after elution was 7.57 wt % (in monocyclic aromatic hydrocarbons, toluene accounted for 12 wt %, ethylbenzene accounted for 36 wt %, and p-xylene accounted for 52 wt %).

Atmospheric and vacuum distillation was performed on column chromatography products to separate toluene from ethylbenzene, p-xylene and high-carbon alkane, to obtain toluene with a purity of 99.57 wt %.

Atmospheric and vacuum distillation was performed on the distillate at the column bottom again to separate ethylbenzene and p-xylene from high-carbon alkane.

Extractive distillation was performed on an enriched mixture of ethylbenzene and p-xylene with 1,2,4-trichlorobenzene, to obtain ethylbenzene with a purity of 98.55 wt %.

Solvent recovery was performed on a mixture of p-xylene and 1,2,4-trichlorobenzene through the solvent recovery column, to obtain p-xylene with a purity of 99.17 wt %.

Table 3 shows the process conditions in Embodiment 2, and Table 4 shows the product compositions of the tower top in Embodiment 2.

TABLE 3
Process conditions in Embodiment 2

| | First | Second | | |
|---|---|---|---|---|
| | atmospheric | atmospheric | | |
| Process | and vacuum | and vacuum | Extractive | Solvent |
| conditions | distillation | distillation | distillation | recovery |
| Quantity of trays | 39 | 22 | 206 | 30 |
| Reflux ratio | 2 | 9 | 37 | 2 |
| Column | 110.3 | 138.2 | 135.7 | 137.8 |
| overhead | | | | |
| temperature/° C. | | | | |
| Column bottom | 171.5 | 177.5 | 164.2 | 202.4 |
| temperature/° C. | | | | |
| Column overhead | 0.11 | 0.11 | 0.11 | 0.11 |
| pressure/MPa | | | | |
| Column bottom | 0.17 | 0.16 | 0.23 | 0.17 |
| pressure/MPa | | | | |

TABLE 4
Composition of the overhead product from Embodiment 2

| | First | Second | | |
|---|---|---|---|---|
| | atmospheric | atmospheric | | |
| | and vacuum | and vacuum | Extractive | Solvent |
| | distillation | distillation | distillation | recovery |
| Composition | column | column | column | column |
| Toluene/wt % | 99.57 | 0.01 | 0.03 | 0.00 |
| Ethylbenzene/wt % | 0.30 | 38.61 | 98.55 | 0.71 |
| P-xylene/wt % | 0.13 | 54.86 | 1.41 | 99.17 |
| High-carbon | 0.00 | 6.52 | 0.00 | 0.12 |
| alkane/wt % | | | | |

### Embodiment 3

Different from Embodiment 1, in the present embodiment, the stationary phase of column chromatography was composed of 3.61 g silica gel for chromatography and 2.54 g neutral aluminum oxide; when performing the elution, 30 mL of eluent 1 and 9 mL of eluent 2 were used to elute in turn, to completely remove low-carbon chain alkanes and cycloalkanes, then 3 mL of eluent 2 was used to elute to obtain a component with monocyclic aromatics content of 14.94 wt % (ethylbenzene accounted for 47 wt % and p-xylene accounted for 53 wt % in monocyclic aromatic hydrocarbons), and the number of atmospheric and vacuum distillation of column chromatography products was 1 time.

Atmospheric and vacuum distillation was performed on the column chromatography products to separate ethylbenzene and p-xylene from high-carbon alkane.

Extractive distillation was performed on a mixture of ethylbenzene and p-xylene with 1,2,4-trichlorobenzene, to obtain ethylbenzene with a purity of 96.03 wt % was obtained.

Solvent recovery was performed on a mixture of p-xylene and 1,2,4-trichlorobenzene through a solvent recovery column, to obtain p-xylene with a purity of 96.69 wt %.

Table 5 shows the process conditions in Embodiment 3, and Table 6 shows the product compositions of the tower top in Embodiment 3.

TABLE 5
Process conditions in Embodiment 3

| | Atmospheric | | |
|---|---|---|---|
| Process | and vacuum | Extractive | solvent |
| conditions | distillation | distillation | recovery |
| Quantity of trays | 35 | 152 | 24 |
| Reflux ratio | 2 | 35 | 3 |
| Column | 137.1 | 135.8 | 137.8 |
| overhead | | | |
| temperature/° C. | | | |
| Column bottom | 208.3 | 196.1 | 214.5 |
| temperature/° C. | | | |
| Column overhead | 0.11 | 0.11 | 0.11 |
| pressure/MPa | | | |
| Column bottom | 0.16 | 0.23 | 0.17 |
| pressure/MPa | | | |

TABLE 6
Composition of the overhead product from Embodiment 3

| | Atmospheric | | |
|---|---|---|---|
| | and vacuum | Extractive | Solvent |
| | distillation | distillation | recovery |
| Composition | column | column | column |
| Ethylbenzene/wt % | 46.83 | 96.03 | 3.12 |
| P-xylene/wt % | 53.15 | 3.97 | 96.69 |
| High-carbon | 0.01 | 0.00 | 0.20 |
| alkane/wt % | | | |

### Embodiment 4

Different from Embodiment 1, in the present embodiment, the stationary phase of column chromatography was composed of 3.76 g silica gel for chromatography and 2.88 g neutral aluminum oxide; when performing the elution, 30 mL of eluent 1 and 6 mL of eluent 2 were used to elute in turn first, to completely remove low-carbon chain alkanes and cycloalkanes, then 3 mL of eluent 2 was used to elute to obtain a component with monocyclic aromatics content of 15.35 wt % (in monocyclic aromatic hydrocarbons, toluene accounted for 24 wt %, ethylbenzene accounted for 26 wt %, and p-xylene accounted for 50 wt %).

Atmospheric and vacuum distillation was performed on the column chromatography products to separate toluene from ethylbenzene and p-xylene, and high-carbon alkane, to obtain toluene with a purity of 99.83 wt %.

Atmospheric and vacuum distillation was performed on the distillate at the column bottom again to separate ethylbenzene and p-xylene from high-carbon alkane.

Extractive distillation was performed on a mixture of ethylbenzene and p-xylene after enrichment with 1,2,4-trichlorobenzene, to obtain ethylbenzene with a purity of 97.99 wt %.

Solvent recovery was performed on a mixture of p-xylene and 1,2,4-trichlorobenzene through the solvent recovery tower, to obtain p-xylene with a purity of 99.42 wt %.

Table 7 shows the process conditions in Embodiment 4, and Table 8 shows the product compositions of the tower top in Embodiment 4.

TABLE 7
Process conditions in Embodiment 4

| | First | Second | | |
|---|---|---|---|---|
| | atmospheric | atmospheric | | |
| Process | and vacuum | and vacuum | Extractive | Solvent |
| conditions | distillation | distillation | distillation | recovery |
| Quantity of trays | 39 | 22 | 207 | 30 |
| Reflux ratio | 2 | 5 | 45 | 3 |
| Column | 110.3 | 137.8 | 135.7 | 137.8 |
| overhead | | | | |
| temperature/° C. | | | | |
| Column bottom | 167.5 | 177.5 | 161.8 | 206.3 |
| temperature/° C. | | | | |
| Column overhead | 0.11 | 0.11 | 0.11 | 0.11 |
| pressure/MPa | | | | |
| Column bottom | 0.17 | 0.16 | 0.23 | 0.17 |
| pressure/MPa | | | | |

TABLE 8
Composition of the overhead product from Embodiment 4

| | First | Second | | |
|---|---|---|---|---|
| | atmospheric | atmospheric | | |
| | and vacuum | and vacuum | Extractive | Solvent |
| | distillation | distillation | distillation | recovery |
| Composition | column | column | column | column |
| Toluene/wt % | 99.83 | 0.03 | 0.09 | 0.00 |
| Ethylbenzene/wt % | 0.11 | 32.89 | 97.99 | 0.52 |
| P-xylene/wt % | 0.06 | 63.55 | 1.91 | 99.42 |
| High-carbon | 0.00 | 3.53 | 0.00 | 0.06 |
| alkane/wt % | | | | |

### Embodiment 5

Different from Embodiment 1, in the present embodiment, the stationary phase of column chromatography was composed of 3.61 g of silica gel for chromatography in the lower layer and 2.88 g of neutral aluminum oxide in the upper layer; when performing the elution, 30 mL of eluent 1 and 9 mL of eluent 2 were used to elute in turn first, to completely remove low-carbon chain alkanes and cycloalkanes, then 3 ml of eluent 2 was used to elute to obtain a component with a content of monocyclic aromatic hydrocarbons as 14.94 wt % (in the monocyclic aromatic hydrocarbons, ethylbenzene accounted for 47 wt % and p-xylene accounted for 53 wt %); and the number of atmospheric and vacuum distillation for column chromatography products was 1 time.

Atmospheric and vacuum distillation was performed on the column chromatography products to separate ethylbenzene and p-xylene from high-carbon alkane.

Extractive distillation was performed on a mixture of ethylbenzene and p-xylene after enrichment with 1,2,4-trichlorobenzene to obtain ethylbenzene with a purity of 99.09 wt %.

The enriched mixture of ethylbenzene and p-xylene was subjected to extractive distillation with 1,2,4-trichlorobenzene, to obtain ethylbenzene with a purity of 99.09 wt %.

Solvent recovery was performed on a mixture of p-xylene and 1,2,4-trichlorobenzene through solvent recovery tower, to obtain p-xylene with a purity of 99.10 wt %.

Table 9 shows the process conditions in Embodiment 5, and Table 8 shows the product compositions of the tower top in Embodiment 5.

TABLE 9
Process conditions in Embodiment 5

| Process | Atmospheric and | Extractive | Solvent |
|---|---|---|---|
| conditions | vacuum distillation | distillation | recovery |
| Quantity of trays | 35 | 215 | 24 |
| Reflux ratio | 3 | 37 | 3 |
| Column | 137.1 | 135.7 | 137.9 |
| overhead | | | |
| temperature/° C. | | | |
| Column bottom | 208.3 | 196.1 | 214.5 |
| temperature/° C. | | | |
| Column | 0.11 | 0.11 | 0.11 |
| overhead | | | |
| pressure/MPa | | | |
| Column bottom | 0.16 | 0.23 | 0.17 |
| pressure/MPa | | | |

TABLE 10
Composition of the overhead product from Embodiment 5

| | | Extractive | Solvent |
|---|---|---|---|
| | Atmospheric and vacuum | distillation | recovery |
| Composition | distillation column | column | column |
| Ethylbenzene/wt % | 46.83 | 99.09 | 0.71 |
| P-xylene/wt % | 53.15 | 0.91 | 99.10 |
| High-carbon | 0.01 | 0.00 | 0.19 |
| alkane/wt % | | | |

It can be seen from the above embodiments that the present disclosure provides a method for separating and purifying monocyclic aromatic hydrocarbons from direct coal liquefaction oil. The present disclosure selects a fraction rich in monocyclic aromatic hydrocarbons after performing fine fractionation and composition analysis on direct coal liquefaction oil, then column chromatography is performed on the selected fractions to remove low-carbon chain alkanes and cycloalkanes, atmospheric and vacuum distillation is performed on components removing low-carbon alkanes to obtain toluene with purity >99 wt % and play the role of enriching ethylbenzene and p-xylene, extractive distillation is used to separate ethylbenzene and p-xylene to obtain ethylbenzene with a purity of >96%, and finally, p-xylene with a purity >99 wt % was obtained by solvent recovery. The present disclosure makes full use of the composition advantages of direct coal liquefaction oil, and can obtain 2-3 kinds of high-purity monomers of monocyclic aromatic hydrocarbons; and the present disclosure does not need to introduce additional chemical reaction steps, and has obvious economic and environmental benefits compared with the catalytic conversion method, which provides a new idea for making full use of coal-based energy.

The above descriptions are only the preferred embodiments of the present disclosure, it is to be pointed out that for ordinary technical personnel in the technical field, without deviating from the principle of the present disclosure, a number of improvements and retouchings can be made, and such improvements and modifications shall fall within the protection scope of the present disclosure.

What is claimed is:

1. A method for separating and purifying a monocyclic aromatic hydrocarbon from a direct coal liquefaction oil, comprising the following steps:
1) performing a fine fractionation on the direct coal liquefaction oil, and analyzing components of obtained fraction sections;
2) selecting a fraction section with a highest content of the monocyclic aromatic hydrocarbon, and sequentially performing a column chromatography, an atmospheric and vacuum distillation, and an extractive distillation on fractions in the fraction section to obtain a distillation product; and
3) performing a solvent recovery on the distillation product to obtain the monocyclic aromatic hydrocarbon;
wherein the monocyclic aromatic hydrocarbon comprises one or more of toluene, ethylbenzene, and p-xylene.

2. The method for separating and purifying the monocyclic aromatic hydrocarbon from the direct coal liquefaction oil according to claim 1, wherein in the step 1), a reflux ratio of the fine fractionation is more than or equal to 3, a temperature range of the fine fractionation is less than or equal to 180° C., and a quantity of the obtained fraction sections is 8-15.

3. The method for separating and purifying the monocyclic aromatic hydrocarbon from the direct coal liquefaction oil according to claim 1, wherein a stationary phase of the column chromatography is a silica gel for chromatography and/or neutral aluminum oxide;
when the stationary phase is a mixture of the silica gel for chromatography and the neutral aluminum oxide, a mass ratio of the silica gel for chromatography to the neutral aluminum oxide is (3-4):(2-3).

4. The method for separating and purifying the monocyclic aromatic hydrocarbon from the direct coal liquefaction oil according to claim 3, wherein in the step 2), a mobile phase comprises a first eluent and a second eluent, and a volume ratio of the first eluent and the second eluent is 10:(3-5);
wherein the first eluent and the second eluent are independently one or more of n-pentane, isopentane, petroleum ether, n-hexane, n-heptane, dichloromethane, ethyl acetate, and chloroform.

5. The method for separating and purifying the monocyclic aromatic hydrocarbon from the direct coal liquefaction oil according to claim 1, wherein in the step 2), after the column chromatography, an obtained column chromatography product is a first mixture, wherein light alkanes and cycloalkanes have been removed from the first mixture.

6. The method for separating and purifying the monocyclic aromatic hydrocarbon from the direct coal liquefaction oil according to claim 5, wherein in the step 2), the atmospheric and vacuum distillation is performed at least once, and an atmospheric and vacuum distillation product is a second mixture, wherein high carbon alkanes have been removed from the second mixture.

7. The method for separating and purifying the monocyclic aromatic hydrocarbon from the direct coal liquefaction oil according to claim 6, wherein during the atmospheric and vacuum distillation, a column overhead temperature is independently 100-140° C., a column bottom temperature is independently 150-220° C., a quantity of trays is independently 20-40, and a reflux ratio is independently 2-5, a column overhead pressure is 0.10-0.15 MPa, and a column bottom pressure is 0.15-0.17 MPa.

8. The method for separating and purifying the monocyclic aromatic hydrocarbon from the direct coal liquefaction oil according to claim 1, wherein in the step 2), an extractant for the extractive distillation is one or more of polychlorobenzenes, anhydrides, esters, ketones, alcohols, and pyridines.

9. The method for separating and purifying the monocyclic aromatic hydrocarbon from the direct coal liquefaction oil according to claim 1, wherein in the step 2), during the extractive distillation, a column overhead temperature is 130-200° C., and a column bottom temperature is 130-200° C., a quantity of trays is 130-200, a reflux ratio is 35-45, a column overhead pressure is 0.10-0.15 MPa, and a column bottom pressure is 0.2-0.25 MPa.

10. The method for separating and purifying the monocyclic aromatic hydrocarbon from the direct coal liquefaction oil according to claim 1, wherein in the step 3), during the solvent recovery, a column overhead temperature is 130-140° C., a column bottom temperature is 195-215° C., a quantity of trays is 24-30, a reflux ratio is 2-3, a column overhead pressure is 0.1-0.15 MPa, and a column bottom pressure is 0.17-0.2 MPa.

11. The method for separation and purifying the monocyclic aromatic hydrocarbon from the direct coal liquefaction oil according to claim 2, wherein a stationary phase of the column chromatography is a silica gel for chromatography and/or neutral aluminum oxide;
when the stationary phase is a mixture of the silica gel for chromatography and the neutral aluminum oxide, a mass ratio of the silica gel for chromatography to the neutral aluminum oxide is (3-4):(2-3).

12. The method for separating and purifying the monocyclic aromatic hydrocarbon from the direct coal liquefaction oil according to claim 11, wherein in the step 2), a mobile phase comprises a first eluent and a second eluent, and a volume ratio of the first eluent and the second eluent is 10:(3-5);
wherein the first eluent and the second eluent are independently one or more of n-pentane, isopentane, petroleum ether, n-hexane, n-heptane, dichloromethane, ethyl acetate, and chloroform.

13. The method for separating and purifying the monocyclic aromatic hydrocarbon from the direct coal liquefaction oil according to claim 2, wherein in the step 2), after the column chromatography, an obtained column chromatography product is a first mixture, wherein light alkanes and cycloalkanes have been removed from the first mixture.

14. The method for separating and purifying the monocyclic aromatic hydrocarbon from the direct coal liquefaction oil according to claim 4, wherein in the step 2), after the column chromatography, an obtained column chromatography product is a first mixture, wherein light alkanes and cycloalkanes have been removed from the first mixture.

15. The method for separating and purifying the monocyclic aromatic hydrocarbon from the direct coal liquefaction oil according to claim 12, wherein in the step 2), after the column chromatography, an obtained column chromatography product is a first mixture, wherein light alkanes and cycloalkanes have been removed from the first mixture.

16. The method for separating and purifying the monocyclic aromatic hydrocarbon from the direct coal liquefaction oil according to claim 13, wherein in the step 2), the atmospheric and vacuum distillation is performed at least once, and an atmospheric and vacuum distillation product is a second mixture, wherein high carbon alkanes have been removed from the second mixture.

17. The method for separating and purifying the monocyclic aromatic hydrocarbon from the direct coal liquefaction oil according to claim 14, wherein in the step 2), the atmospheric and vacuum distillation is performed at least once, and an atmospheric and vacuum distillation product is a second mixture, wherein high carbon alkanes have been removed from the second mixture.

18. The method for separating and purifying the monocyclic aromatic hydrocarbon from the direct coal liquefaction oil according to claim 15, wherein in the step 2), the atmospheric and vacuum distillation is performed at least once, and an atmospheric and vacuum distillation product is a second mixture, wherein high carbon alkanes have been removed from the second mixture.

19. The method for separating and purifying the monocyclic aromatic hydrocarbon from the direct coal liquefaction oil according to claim 16, wherein during the atmospheric and vacuum distillation, a column overhead temperature is independently 100-140° C., a column bottom temperature is independently 150-220° C., a quantity of trays is independently 20-40, and a reflux ratio is independently 2-5, a column overhead pressure is 0.10-0.15 MPa, and a column bottom pressure is 0.15-0.17 MPa.

20. The method for separating and purifying the monocyclic aromatic hydrocarbon from the direct coal liquefaction oil according to claim 17, wherein during the atmospheric and vacuum distillation, a column overhead temperature is independently 100-140° C., a column bottom temperature is independently 150-220° C., a quantity of trays is independently 20-40, and a reflux ratio is independently 2-5, a column overhead pressure is 0.10-0.15 MPa, and a column bottom pressure is 0.15-0.17 MPa.
